(19) United States
(12) Patent Application Publication
(10) Pub. No.: US 2023/0047488 A1
(43) Pub. Date: Feb. 16, 2023

(54) PROCESS FOR THE COSMETIC TREATMENT OF KERATIN FIBRES USING A DETERGENT COMPOSITION AND A RARE EARTH METAL COMPOSITION

(71) Applicant: L'OREAL, Paris (FR)

(72) Inventors: Valérie JEANNE-ROSE, AULNAY-SOUS-BOIS (FR); Henri SAMAIN, CHEVILLY-LARUE (FR); Laurent SABATIE, AULNAY-SOUS-BOIS (FR)

(73) Assignee: L'OREAL, Paris (FR)

(21) Appl. No.: 17/787,247

(22) PCT Filed: Dec. 17, 2020
(86) PCT No.: PCT/EP2020/086786

§ 371 Date: Jun. 17, 2022

(30) Foreign Application Priority Data
Dec. 20, 2019 (FR) 1915317

(51) Int. Cl. A61K 8/46; A61K 8/19; A61K 8/41; A61K 8/34; A61Q 5/00

(52) U.S. Cl. CPC A61K 8/466; A61K 8/19; A61K 8/41; A61K 8/342; A61Q 5/002; A61K 2800/48

(57) ABSTRACT

The invention relates to a process for the cosmetic treatment of keratin fibres, in particular human keratin fibres such as the hair, implementing the application of a composition comprising at least one surfactant, and the application of a composition comprising a particular metal compound and a conditioning agent chosen from cationic surfactants and/or fatty alcohols.

The invention relates to a process for the cosmetic treatment of keratin fibres, in particular human keratin fibres such as the hair, implementing the application of a composition comprising at least one surfactant, and the application of a composition comprising a particular metal compound and a conditioning agent chosen from cationic surfactants and/or fatty alcohols.

The present invention also relates to a multi-compartment kit or device containing said composition comprising at least one surfactant and said composition comprising a metal compound.

For washing and conditioning keratin fibres such as the hair, it is common practice to use a multistep process using a detergent composition (such as a shampoo) and then a conditioning composition (such as a conditioner care product). This process requires performing an intermediate rinsing operation between the application of the detergent composition and the application of the conditioning composition, before carrying out a final rinsing operation after the application of the conditioning composition.

However, the need to carry out an intermediate rinsing operation represents a constraint and wasted time for consumers. It also represents a not insignificant environmental impact due to a considerable water consumption.

Moreover, the existing conditioning compositions do not have cosmetic properties that are satisfactory for consumers. This is because they are capable of weighing down the hair or leaving a feel that can be perceived by the user as greasy and consequently rather unpleasant and that is not what the user is looking for.

There is therefore a real need to develop a process which allies washing and conditioning and which offers a considerable time saving while at the same time exhibiting good washing properties and good cosmetic properties, notably in terms of conditioning and providing body.

It has been discovered, surprisingly, that a three-step cosmetic treatment process comprising the application, without intermediate rinsing, of a composition comprising at least one rare earth metal after the application of a composition comprising at least one surfactant makes it possible to achieve the objectives above, notably in terms of washing and conditioning the hair and giving it body. Moreover, this process has the advantage of considerably reducing the consumption of water required for washing the hair.

A subject of the invention is therefore a process for the cosmetic treatment of keratin fibres, comprising:

i) a step of applying to said keratin fibres a composition (A) comprising one or more surfactants chosen from anionic surfactants and amphoteric or zwitterionic surfactants, and mixtures thereof,

ii) a step of applying to said keratin fibres a composition (B) comprising one or more compounds of a metal belonging to the group of the rare earth metals, and one or more conditioning agents chosen from cationic surfactants and fatty alcohols, and mixtures thereof, then

iii) a rinsing step.

According to one embodiment of the invention, the composition (A) and the composition (B) are applied simultaneously to the keratin fibres.

According to another embodiment, the composition (B) is applied to the keratin fibres directly after applying the composition (A), without an intermediate step of rinsing said keratin fibres.

The process according to the invention makes it possible to give the keratin fibres satisfactory washing effects, and also good conditioning properties, notably easy disentangling, and supplementary properties, notably in terms of providing body and volume.

Furthermore, despite the absence of intermediate rinsing between steps i) (washing) and ii) (conditioning), the conditioning properties introduced by step ii) are preserved.

Thus, the process according to the invention results in satisfactory conditioning while at the same time reducing the treatment time and minimizing the amount of water required.

A subject of the invention is also a multi-compartment kit or device comprising a first compartment containing a detergent composition (A) as defined above, and a second compartment containing a composition (B) as defined above.

Other subjects, features, aspects and advantages of the invention will emerge even more clearly on reading the description and the examples that follow.

In the text hereinbelow, and unless otherwise indicated, the limits of a range of values are included in that range, notably in the expressions “between” and “ranging from . . . to . . . ”.

Moreover, the expression “at least one” used in the present description is equivalent to the expression “one or more”.

### COMPOSITION (A)

The process according to the present invention comprises a step i) consisting in applying to the keratin fibres, in particular human keratin fibres such as the hair, a composition (A) comprising one or more surfactants chosen from anionic surfactants, amphoteric or zwitterionic surfactants, and mixtures thereof.

According to one particular embodiment, this composition (A) is such that the total amount of surfactants chosen from the list below is at least 4% by weight, of the total weight of the composition.

According to this embodiment, this gives a detergent composition.

According to the present invention, the term “detergent composition” is intended to mean a composition intended for washing keratin fibres, i.e. a composition which makes it possible to remove from said keratin fibres, notably the hair, the soiling adhering thereto by placing said fibres in dispersion or in solution.

According to one detergent composition variant, the composition (A) comprises one or more anionic and/or amphoteric surfactants, preferably in a total content of greater than 4% by weight, relative to the total weight of the composition.

### Anionic Surfactants

The composition (A) used in step i) of the process for the treatment of keratin fibres according to the present invention can comprise one or more anionic surfactants.

The term “anionic surfactant” is intended to mean a surfactant comprising, as ionic or ionizable groups, only anionic groups.

In the present description, an entity is described as being “anionic” when it bears at least one permanent negative charge or when it can be ionized as a negatively charged entity, under the conditions of use of the composition of the invention (for example the medium or the pH) and not comprising any cationic charge.

The anionic surfactants may be notably chosen from sulfate, sulfonate and carboxylic (or carboxylate) surfactants. Needless to say, a mixture of these surfactants may be used.

It is understood in the present description that:

the carboxylate anionic surfactants comprise at least one carboxylic or carboxylate function (—COOH or —COO⁻) and may optionally also comprise one or more sulfate and/or sulfonate functions;

the sulfonate anionic surfactants comprise at least one sulfonate function (—SO₃H or —SO₃⁻) and may optionally also comprise one or more sulfate functions, but do not comprise any carboxylate functions; and

the sulfate anionic surfactants comprise at least one sulfate function but do not comprise any carboxylate or sulfonate functions.

The carboxylic anionic surfactants that may be used thus include at least one carboxylic or carboxylate function (—COOH or —COO⁻).

The carboxylic anionic surfactants may be chosen from the following compounds: acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds;

the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Use may also be made of C₆-C₂₄ alkyl monoesters of polyglycoside-polycarboxylic acids such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Among the above carboxylic surfactants, mention may be made most particularly of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names.

The polyoxyalkylenated alkyl(amido) ether carboxylic acids that may be used are preferably chosen from those of formula (1):

R₁ [symbol] OC₂H₄)ₙ—OCH₂COOA (1)

in which:

R₁ represents a linear or branched C₆-C₂₄ alkyl or alkenyl radical, a (C₈-C₉)alkylphenyl radical, a radical R₂CONH—CH₂—CH₂— with R₂ denoting a linear or branched C₉-C₂₁ alkyl or alkenyl radical;

preferably, R₁ is a C₈-C₂₀ and preferably C₈-C₁₈ alkyl radical, and aryl preferably denotes phenyl,

n is an integer or decimal number (mean value) ranging from 2 to 24 and preferably from 2 to 10,

A denotes H, ammonium, Na, K, Li, Mg or a monoethanolamine or triethanolamine residue.

It is also possible to use mixtures of compounds of formula (1), in particular mixtures of compounds containing different groups R1.

The polyoxyalkylenated alkyl(amido) ether carboxylic acids that are particularly preferred are those of formula (1) in which:

R₁ denotes a C₁₂-C₁₄ alkyl, cocoyl, oleyl, nonylphenyl or octylphenyl radical,

A denotes a hydrogen or sodium atom, and

n ranges from 2 to 20, preferably from 2 to 10.

Even more preferentially, use is made of the compounds of formula (1) in which R denotes a C₁₂ alkyl radical, A denotes a hydrogen or sodium atom and n ranges from 2 to 10.

Preferentially, the carboxylic anionic surfactants are chosen, alone or as a mixture, from:

acylglutamates, notably of C₆-C₂₄ or even C₁₂-C₂₀, such as stearoylglutamates, and in particular disodium stearoylglutamate;

acylsarcosinates, notably of C₆-C₂₄ or even C₁₂-C₂₀, such as palmitoylsarcosinates, and in particular sodium palmitoylsarcosinate;

acyllactylates, notably of Cu-Cm or even C₁₄-C₂₄, such as behenoyllactylates, and in particular sodium behenoyllactylate;

C₆-C₂₄ and notably C₁₂-C₂₀ acylglycinates;

(C₆-C₂₄)alkyl ether carboxylates, and notably (C₁₂-C₂₀)alkyl ether carboxylates;

polyoxyalkylenated (C₆-C₂₄)alkyl(amido) ether carboxylic acids, in particular those including from 2 to 50 ethylene oxide groups;

in particular in the form of alkali metal or alkaline-earth metal, ammonium or amino alcohol salts.

The sulfonate anionic surfactants that may be used include at least one sulfonate function (—SO₃H or —SO₃⁻).

They may be chosen from the following compounds: alkylsulfonates, alkyl amidesulfonates, alkylarylsulfonates, a-olefin sulfonates, paraffin sulfonates, alkyl sulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Preferentially, the sulfonate anionic surfactants are chosen, alone or as a mixture, from:

C₆-C₂₄ and notably C₁₂-C₂₀ alkyl sulfosuccinates, notably lauryl sulfosuccinates;

C₆-C₂₄ and notably C₁₂-C₂₀ alkyl ether sulfosuccinates;

(C₆-C₂₄)acylisethionates, preferably (C₁₂-C₁₈)acylisethionates;

in particular in the form of alkali metal or alkaline-earth metal, ammonium or amino alcohol salts.

The sulfate anionic surfactants that may be used include at least one sulfate function (—OSO₃H or —OSO₃⁻).

They may be chosen from the following compounds: alkyl sulfates, alkyl ether sulfates, alkylamido ether sulfates, alkylaryl polyether sulfates, monoglyceride sulfates; and the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, in particular polyoxyethylenated, and then preferably comprising from 1 to 50 ethylene oxide units and better still from 2 to 10 ethylene oxide units.

Preferentially, the sulfate anionic surfactants are chosen, alone or as a mixture, from:

alkyl sulfates, notably C₆-C₂₄ or even C₁₂-C₂₀ alkyl sulfates;

alkyl ether sulfates, notably C₆-C₂₄ or even C₁₂-C₂₀ alkyl ether sulfates, preferably comprising from 2 to 20 ethylene oxide units;

in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Among the anionic surfactants that may be used according to the invention, mention may also be made of phosphoric surfactants.

The term “phosphoric surfactant” means a surfactant of which the polar part comprises at least one phosphorus atom.

The phosphoric surfactant may have the following formula:

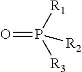

in which

R₁, R₂ and R₃, which may be identical or different, represent a group chosen from:

a group —OM, in which M represents a hydrogen atom or an alkali metal, such as Na, Li or K, preferably Na or K;

a group —OR₄, in which R₄ represents a linear or branched C₁-C₄₀ alkyl group, preferably a C₁₂-C₂₀ alkyl group and more preferably a C₁₆ or C₁₈ alkyl group, a linear or branched C₂-C₄₀ alkenyl group, preferably a C₁₂-C₂₀ alkenyl group and more preferably a C₁₆ or C₁₈ alkenyl group, a C₃-C₄₀ cyclic alkyl group, a C₃-C₄₀ cyclic alkenyl group, a C₅-C₄₀ aromatic group or a C₆-C₄₀ aralkyl group; and

an oxyalkylene group

—OCH₂CH₂)ₙ(OCH₂CH(CH₃))ₘOR₄ in which R₄ is as defined previously, n represents an integer ranging from 1 to 50 and m represents an integer ranging from 0 to 50,

given that at least one from among R₁, R₂ and R₃ is a group

—OM and that at least one from among R₁, R₂ and R₃ is a group —OR₄ or

—(OCH₂CH₂)ₙ(OCH₂CH(CH₃))ₘOR₄.

Preferably, the phosphoric surfactant may be chosen from oxyalkylenated C₁₂-C₂₀ alcohol phosphates containing from 1 to 50 mol of alkylene oxide chosen from ethylene oxide and propylene oxide, and non-oxyalkylenated C₁₂-C₂₀ alcohol dialkyl phosphates, and mixtures thereof. The alkyl groups of the oxyalkylenated alcohol and/or of the non-oxyalkylenated alcohol may be linear or branched, and saturated or unsaturated.

Preferably, use may be made of a combination of at least one oxyalkylenated phosphoric surfactant and of at least one non-oxyalkylenated phosphoric surfactant.

More preferably, the combination of phosphoric surfactants may be chosen from the group constituted of a combination of ceteth-10 phosphate and dicetyl phosphate, a combination of ceteth-20 phosphate and dicetyl phosphate, and a combination of oleth-5 phosphate and dioleyl phosphate.

As product comprising the combination of ceteth-10 phosphate and dicetyl phosphate, mention may be made of Crodafos CES or Crodafos CES-PA, sold by Croda. As product comprising the combination of ceteth-20 phosphate and dicetyl phosphate, mention may be made of Crodafos CS-20 Acid, sold by Croda. As product comprising the combination of oleth-5 phosphate and dioleyl phosphate, mention may be made of Crodafos HCE, sold by Croda.

According to one preferred embodiment of the invention, the composition (A) comprises one or more anionic surfactants; more preferentially, the anionic surfactant(s) are chosen from, alone or as a mixture:

C₆-C₂₄ and notably C₁₂-C₂₀ alkyl sulfates,

C₆-C₂₄ and notably C₁₂-C₂₀ alkyl ether sulfates; preferably comprising from 2 to 20 ethylene oxide units;

C₆-C₂₄ and notably C₁₂-C₂₀ alkyl sulfosuccinates, notably lauryl sulfosuccinates;

C₆-C₂₄ and notably C₁₂-C₂₀ alkyl ether sulfosuccinates;

(C₆-C₂₄)acylisethionates, preferably (C₁₂-C₁₈)acylisethionates;

C₆-C₂₄ and notably C₁₂-C₂₀ acylsarcosinates; notably palmitoylsarcosinates;

(C₆-C₂₄)alkyl ether carboxylates, preferably (C₁₂-C₂₀)alkyl ether carboxylates;

polyoxyalkylenated (C₆-C₂₄)alkyl(amido) ether carboxylic acids and salts thereof, in particular those including from 2 to 50 alkylene oxide and in particular ethylene oxide groups;

C₆-C₂₄ and notably C₁₂-C₂₀ acylglutamates;

C₆-C₂₄ and notably C₁₂-C₂₀ acylglycinates;

in particular in the form of alkali metal or alkaline-earth metal, ammonium or amino alcohol salts.

Advantageously, the amount of anionic surfactants in the composition (A) can range between 0 and 30% by weight, relative to the total weight of the composition (A).

### Amphoteric or Zwitterionic Surfactants

The composition (A) used in step i) of the process for the treatment of keratin fibres according to the present invention can comprise one or more amphoteric or zwitterionic surfactants.

The amphoteric or zwitterionic surfactant(s) that may be used according to the invention may be optionally quaternized secondary or tertiary aliphatic amine derivatives, in which the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, (C₈-C₂₀)alkyl sulfobetaines, (C₈-C₂₀)alkylamido(C₃-C₈)alkylbetaines and (C₈-C₂₀)alkylamido(C₆-C₈)alkyl sulfobetaines.

Among the optionally quaternized derivatives of secondary or tertiary aliphatic amines that may be used, as defined above, mention may also be made of the compounds having the respective formulae (3) and (4) below:

Rₐ—CONHCH₂CH₂—N⁺(R_{b})(R_{c})—CH₂COO⁻,M⁺,X⁻ (3)

in which:

Rₐ represents a C₁₀ to C₃₀ alkyl or alkenyl group derived from an acid RₐCOOH preferably present in hydrolyzed coconut kernel oil, or a heptyl, nonyl or undecyl group;

R_{b} represents a β-hydroxyethyl group; and

R_{c} represents a carboxymethyl group;

M⁺ represents a cationic counterion derived from an alkali metal or alkaline-earth metal, such as sodium, an ammonium ion or an ion derived from an organic amine; and

X⁻ represents an organic or mineral anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M⁺ and X⁻ are absent;

R_{a′}—CONHCH₂CH₂—N(B)(B′) (4)

in which:

B represents the group —CH₂CH₂OX′;

B′ represents the group —(CH₂)_{z}Y′, with z=1 or 2;

X′ represents the group —CH₂COOH, —CH₂—COOZ′, —CH₂CH₂COOH or CH₂CH₂-COOZ′, or a hydrogen atom;

Y′ represents the group —COOH, —COOZ′ or —CH₂CH(OH)SO₃H or the group CH₂CH(OH)SO₃—Z′;

Z′ represents a cationic counterion derived from an alkali metal or alkaline-earth metal, such as sodium, an ammonium ion or an ion derived from an organic amine; and

R_{a′} represents a C₁₀ to C₃₀ alkyl or alkenyl group of an acid Rₐ—COOH which is preferably present in coconut kernel oil or in hydrolyzed linseed oil, or an alkyl group, in particular a C₁₇ group, and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of the compounds of formula (5):

R_{a″}—NHCH(Y″)—(CH₂)ₙCONH(CH₂)_{n′}—N(R_{d})(Rₑ) (5)

in which:

Y″ represents the group —COOH, —COOZ″ or —CH₂—CH(OH)SO₃H or the group CH₂CH(OH)SO₃—Z″;

R_{d} and Rₑ, independently of one another, represent a C₁ to C₄ alkyl or hydroxyalkyl radical;

Z″ represents a cationic counterion derived from an alkali metal or alkaline-earth metal, such as sodium, an ammonium ion or an ion derived from an organic amine;

R_{a″} represents a C₁₀ to C₃₀ alkyl or alkenyl group of an acid R_{a″}—COOH which is preferably present in coconut kernel oil or in hydrolysed linseed oil; and

n and n′ denote, independently of one another, an integer ranging from 1 to 3.

Among the compounds of formula (5), mention may be made of the compound classified in the CTFA dictionary under the name sodium diethylaminopropyl cocoaspartamide and sold by the company Chimex under the name Chimexane HB.

These compounds may be used alone or as mixtures.

Preferably, the amphoteric or zwitterionic surfactant(s) are chosen from (C₈-C₂₀)alkylbetaines, (C₈-C₂₀)alkyl sulfobetaines, (C₈-C₂₀)alkylamido(C₃-C₈)alkylbetaines and (C₈-C₂₀)alkylamido(C₆-C₈)alkylsulfobetaines, the compounds of formulae (3), (4) and (5) as defined previously; and mixtures thereof.

Preferentially, the amphoteric or zwitterionic surfactant(s) are chosen from (C₈-C₂₀)alkylbetaines such as cocoylbetaine, (C₈-C₂₀)alkylamido(C₃-C₈)alkylbetaines such as cocamidopropylbetaine, or the compounds of formula (3) such as disodium cocoamphodiacetate, and mixtures thereof.

More preferentially, the amphoteric or zwitterionic surfactant(s) are chosen from (C₈-C₂₀)alkylbetaines such as cocoylbetaine, (C₈-C₂₀)alkylamido(C₃-C₈)alkylbetaines such as cocamidopropylbetaine, and mixtures thereof.

According to one preferred embodiment of the invention, the composition (A) comprises one or more amphoteric or zwitterionic surfactants; preferentially, the amphoteric or zwitterionic surfactant(s) are chosen from (C₈-C₁₆)alkylamido(C₃)alkylbetaine and in particular cocoamidopropylbetaine.

The amount of amphoteric surfactants can range between 0 and 25% by weight, relative to the total weight of the composition (A).

Advantageously, the total content of the surfactant(s) chosen from anionic surfactants and amphoteric or zwitterionic surfactants, and mixtures thereof, is greater than or equal to 4% by weight, preferably ranges from 4% to 35% by weight, relative to the total weight of the composition (A).

According to one embodiment, the composition (A) comprises one or more anionic surfactants in an amount of between 4% and 25% by weight, of the total weight of the composition, and optionally one or more amphoteric surfactants.

According to another embodiment, the composition (A) comprises one or more amphoteric surfactants in an amount of between 4% and 25% by weight, of the total weight of the composition, and optionally one or more anionic surfactants.

According to another embodiment, the composition comprises at least one anionic surfactant and at least one amphoteric surfactant, the total amount of anionic and amphoteric surfactants being between 4% and 35%, preferably between 4% and 25% by weight, of the total weight of the composition.

The composition (A) is preferably aqueous or aqueous-alcoholic.

When the composition (A) is aqueous, it preferably comprises water in a content of greater than or equal to 50% by weight, more preferentially greater than or equal to 70% by weight, even more preferentially still greater than or equal to 90% by weight, relative to the total weight of the composition (A).

When the composition (A) is aqueous-alcoholic, it's can notably comprise one or more organic solvents, preferably in a content ranging from 0.05% to 95% by weight, and more preferentially from 1% to 70% by weight, relative to the total weight of the composition (A).

This organic solvent may be a C₂ to C₄ lower alcohol, in particular ethanol and isopropanol, polyols and polyol ethers such as propylene glycol, polyethylene glycol or glycerol. The organic solvent is preferably ethanol.

### COMPOSITION (B)

The process according to the present invention also comprises a step ii) consisting in applying to the keratin fibres, in particular human keratin fibres such as the hair, a composition (B) comprising one or more compounds of a metal belonging to the group of the rare earth metals, and one or more conditioning agents, preferably chosen from cationic surfactants and/or fatty alcohols, and mixtures thereof.

Step ii) follows step i). In other words, the composition (A), as defined above, is applied before the composition (B).

The composition (B) used in step ii) of the process according to the invention is preferably non-detergent.

For the purposes of the present invention, the term “non-detergent” composition means a composition not intended for washing keratin fibres, i.e. a composition which does not make it possible to remove from said keratin fibres, notably the hair, the soiling adhering thereto by placing said fibres in dispersion or in solution.

According to this particular embodiment, the composition (B) contains little or no anionic and/or amphoteric surfactants. Preferably, if this composition (B) contains anionic and/or amphoteric surfactants, the composition (B) according to the invention contains less than 4% by total weight of anionic and amphoteric or zwitterionic surfactants, relative to the total weight of the composition. According to one particularly preferred embodiment, the composition (B) contains less than 1% of anionic surfactants and of amphoteric or zwitterionic surfactants; it preferably does not contain any of said surfactants.

### Compounds of a Metal Belonging to the Group of the Rare Earth Metals

The composition (B) used in step ii) of the process for the treatment of keratin fibres according to the present invention comprises one or more compounds of a metal belonging to the group of the rare earth metals (rare earth metal compound). By way of example of a metal belonging to the group of the rare earth metals M, mention may be made of scandium, yttrium, lanthanum, cerium, praseodymium, neodymium, promethium, samarium, europium, gadolinum, terbium, dysprosium, holmium, erbium, thulium, ytterbium and lutecium.

Preferably, the metal(s) belonging to the group of the rare earth metals are chosen from cerium, yttrium, ytterbium, lanthanum and europium.

According to one particular embodiment, the metal belonging to the group of the rare earth metals is in +III form.

According to the invention, the compound of a metal belonging to the group of the rare earth metals is chosen from rare earth metal salts, rare earth metal complexes or rare earth metal oxides.

The rare earth metal salts according to the invention may be soluble or insoluble in the composition containing them.

### Definition of the Rare Earth Metal Salts and Complexes

In the case of the rare earth metal salts and complexes according to the invention, the compound of a metal belonging to the group of the rare earth metals is in the +III oxidation state.

The metal M can then be combined, via its electron shell, with n1 anionic groups forming an ionic bond with M and/or with n2 groups forming a coordination bond with M. The groups forming a coordination bond are groups having a donor pair, such as carbonyl or amine, for example.

If n2=0, the compound of a metal belonging to the group of the rare earth metals forms a salt and, in this case, the metal M is combined with 3 anionic groups.

If n2>0, the compound of a metal belonging to the group of the rare earth metals forms a complex and, in this case, the number of anionic groups n1 can vary from 0 to 3.

The metal M is combined with one or more anionic groups and/or one or more groups forming a coordination bond.

Subsequently, the term “ligand” is understood to mean an ion or a molecule carrying a group which is combined, via an ionic bond or a coordination bond, with the metal M. One and the same ligand can carry several groups.

The term “rare earth metal compound” is understood to mean the combination of the metal M with its ligand(s).

A definition of the salts or complexes of rare earth metals can be found in the document: Progress in the Science and Technology of the Rare Earths, Volume 1, edited by Leroy Eyring in 1964, published by Macmillan Company and written by F. Gaume-Mahn, page 259 et seq.

### Ligands Combined with the Rare Earth Metals M to Form a Salt or a Complex and Corresponding Rare Earth Metal Compounds

Typically, the ligand can be a monoatomic or non-monoatomic monoanionic ion, such as a nitrate or a hydroxide (OH⁻) or a halide (typically chloride or bromide). By way of example, the rare earth metal compound which results therefrom can then be MCl₃, M(OH)₃, M(NO₃)₃ and the like, and in particular CeNO₃, YNO₃, LaNO₃, CeCl₃, YCl₃ or LaCl₃.

The ligand can be a di- or trianionic ion, such as phosphate or sulfate. Mention may be made, by way of example, of the rare earth metal compounds such as MPO₄ or M₂(SO₄)₃ and in particular CePO₄, YPO₄, LaPO₄, Ce₂(SO₄)₃, Y₂(SO₄)₃ or La₂(SO₄)₃.

The ligand can contain one or more groups forming a coordination bond and a function forming an ionic bond.

Thus, the ligand can be a mono- or polycarboxylate molecule, such as acetate or succinate. In this case, it is considered that the carboxylate function acts as anionic group, via the hydroxyl of the carboxyl group, and acts as group forming a coordination bond, via the doublet of the oxygen of the carbonyl function. Thus, the resulting rare earth metal compound can be M(R—(COO)ₙ)_{3/n}. The ligand can, in addition to carrying one or more carboxylates, comprise other functions, such as hydroxyls or amines. Thus, the ligand can be to use hydroxycarboxylic acids or aminocarboxylic acids. Mention may be made, as mono- or polycarboxylic compound carrying additional functions, of tartrate, citrate, glycolate or ethylenediaminetetraacetate (EDTA) ions.

The ligand can carry a non-localized anionic charge, such as, for example, acetylacetonate. The rare earth metal compounds will then be M(acetylacetonate)₃ or M(acetylacetonate)₃.7H₂O where each acetonate is bonded to the metal M by its two carbonyl functions, one acting as anionic group and the other as group which is bonded by coordination.

It can also be of the aromatic type, such as a phenol, a cyclopentadiene (Progress in the Science and Technology of the Rare Earths, edited by Leroy Eyring and written by F. Gaume-Mahn, page 296) or a pyridine.

The rare earth metal compound can comprise one or more ligands forming a coordination bond and one or more ligands forming an ionic bond. Thus, the rare earth metal compound can be yttrium dihydroxyacetate Y((OH)₂acetate) (Synthesis and Properties of Yttrium Hydroxyacetate Sols by S. S. Balabanov, E. M Gavrishchuk and D. A. Permin, published in the review Inorganic Materials, 2012, Vol. 48, No. 5, pp. 500-503, in 2012).

The rare earth metal compound can be a double salt, for example with a rare earth metal M and another cation different from the rare earth metals, such as, for example, an alkali metal (Li,Ce(SO₄)₂) or an alkaline earth metal or an organic cationic entity, such as a quaternary amine, such as an alkylpyridinium group.

Often very hygroscopic, these rare earth metal compounds can be found in the form of hydrates, such as, for example, CeCl₃.7H₂O, YCl₃.6H₂O, LaCl₃.7H₂O or Ce(acetonate)₃.xH₂O.

### Rare Earth Metal Oxides

Oxides of rare earth metals are known from the prior art.

In the case of the oxides of rare earth metals according to the invention, the compound of a metal belonging to the group of the rare earth metals is in the +IV oxidation state in the specific case of cerium oxides and in the +III oxidation state for the other rare earth metals as defined above.

Mention may be made, by way of oxide, of the oxides of cerium (CeO₂), of lanthanum (La₂O₃), of yttrium (Y₂O₃), of erbium (Er₂O₃), of scandium (Sc₂O₃), of ytterbium (Yb₂O₃) or of europium (Eu₂O₃), preferably the oxides of cerium and of lanthanum. Preferably, the oxides of rare earth metals of use are cerium oxide, lanthanum oxide and yttrium oxide.

Preferably, the compound of a metal belonging to the group of the rare earth metals is dissolved in the composition (B).

Advantageously, the content of the metal compound(s) is greater than or equal to 0.1% by weight, preferably greater than or equal to 0.25% by weight, more preferentially ranges from 0.5% to 20% by weight, relative to the total weight of the composition (B).

Preferably, the solution B has a pH of between 2 and 8.

### Cationic Surfactants

Preferably, the composition (B) used in step ii) of the process for the treatment of keratin fibres according to the present invention also comprises one or more conditioning agents that can be chosen from cationic surfactants.

The term “cationic surfactant” is intended to mean a surfactant that is positively charged when it is contained in the compositions according to the invention. This surfactant may bear one or more positive permanent charges or may contain one or more cationizable functions in the compositions according to the invention.

The cationic surfactants are advantageously chosen from optionally polyoxyalkylenated primary, secondary or tertiary fatty amine salts; quaternary ammonium salts, and mixtures thereof.

As quaternary ammonium salts, mention may be made notably of:

the quaternary ammonium salts of formula (Ia):

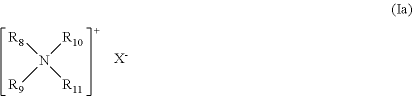

in which:

the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ containing from 8 to 30 and preferably from 12 to 24 carbon atoms; it is possible for the aliphatic groups to comprise heteroatoms such as, in particular, oxygen, nitrogen, sulfur and halogens; and

X⁻ is an anion chosen notably from the group of the halides, phosphates, acetates, lactates, (C₁-C₄)alkyl sulfates, and (C₁-C₄)alkylsulfonates or (C₁-C₄)alkylarylsulfonates.

The aliphatic groups R₈ to R₁₁ may be chosen from C₁-C₃₀ alkyl, C₁-C₃₀ alkoxy, (C₂-C₆) polyoxyalkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups.

Mention may be made notably of tetraalkylammonium halides, notably chlorides, such as dialkyldimethylammonium or alkyltrimethylammonium chlorides in which the alkyl group includes from 12 to 22 carbon atoms, in particular behenyltrimethylammonium chloride, distearyldimethylammonium chloride, cetyltrimethylammonium chloride and benzyldimethylstearylammonium chloride.

Mention may also be made of palmitylamidopropyltrimethylammonium or stearamidopropyldimethyl-(myristyl acetate)-ammonium halides, and notably chlorides; notably the product sold under the name Ceraphyl® 70 by the company Van Dyk.

the quaternary ammonium salts of imidazoline of formula (IIa):

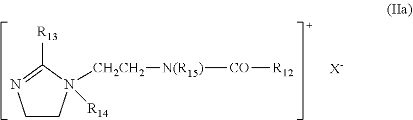

in which:

R₁₂ represents an alkenyl or alkyl group including from 8 to 30 carbon atoms, for example derived from tallow fatty acids,

R₁₃ represents a hydrogen atom, a C₁-C₄ alkyl group or an alkenyl or alkyl group including from 8 to 30 carbon atoms,

R₁₄ represents a C₁-C₄ alkyl group,

R₁₅ represents a hydrogen atom or a C₁-C₄ alkyl group,

X⁻ is an anion chosen notably from the group of the halides, phosphates, acetates, lactates, (C₁-C₄)alkyl sulfates, and (C₁-C₄)alkylsulfonates or (C₁-C₄)alkylarylsulfonates.

Preferably, R₁₂ and R₁₃ denote a mixture of alkenyl or alkyl groups including from 12 to 21 carbon atoms, for example derived from tallow fatty acids, R₁₄ denotes a methyl group and R₁₅ denotes a hydrogen atom. Such a product is sold, for example, under the name Rewoquat® W75 or W90 by the company Evonik.

the quaternary di- or triammonium salts of formula (IIIa):

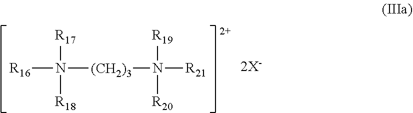

in which:

R₁₆ denotes an alkyl group including from 16 to 30 carbon atoms, which is optionally hydroxylated and/or optionally interrupted with one or more oxygen atoms,

R₁₇ denotes hydrogen, an alkyl group including from 1 to 4 carbon atoms or a group —(CH₂)₃—N⁺(R₁₆ₐ)(R₁₇ₐ)(R₁₈ₐ); R₁₆ₐ, R₁₇ₐ and R₁₈ₐ, which may be identical or different, denoting hydrogen or an alkyl group including from 1 to 4 carbon atoms,

R₁₈, R₁₉, R₂₀ and R₂₁, which may be identical or different, denote hydrogen or an alkyl group including from 1 to 4 carbon atoms, and

X⁻ is an anion, chosen notably from the group of the halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P (Quaternium 89) and Finquat CT (Quaternium 75), sold by the company Finetex.

quaternary ammonium salts containing one or more ester functions, of formula (IVa) below:

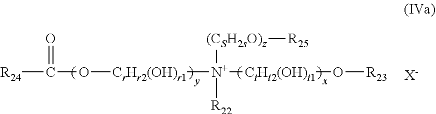

in which:

R₂₂ is chosen from C₁-C₆ alkyl groups and C₁-C₆ hydroxyalkyl or dihydroxyalkyl groups,

R₂₃ is chosen from the group R₂₆—C(═O)—; linear or branched, saturated or unsaturated C₁-C₂₂ hydrocarbon-based groups R₂₇; and a hydrogen atom,

R₂₅ is chosen from the group R₂₈—C(═O)—; linear or branched, saturated or unsaturated C₁-C₆ hydrocarbon-based groups R₂₉; and a hydrogen atom,

R₂₄, R₂₆ and R₂₈, which may be identical or different, are chosen from linear or branched, saturated or unsaturated C₇-C₂₁ hydrocarbon-based groups,

r, s and t, which may be identical or different, are integers ranging from 2 to 6,

r1 and t1, which may be identical or different, are equal to 0 or 1,

y is an integer ranging from 1 to 10,

x and z, which may be identical or different, are integers ranging from 0 to 10,

X⁻ is an anion,

it being understood that r2+r1=2r and t1+t2=2t, and that the sum x+y+z ranges from 1 to 15,

with the proviso that when x=0 then R₂₃ denotes R₂₇ and that when z=0 then R₂₅ denotes R₂₉.

The alkyl groups R₂₂ may be linear or branched, preferably linear. Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may comprise from 12 to 22 carbon atoms, or else may comprise from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide, preferably chloride, bromide or iodide, a (C₁-C₄)alkyl sulfate, a (C₁-C₄)alkylsulfonate or a (C₁-C₄)alkylarylsulfonate, a methanesulfonate, a phosphate, a nitrate, a tosylate, an anion derived from organic acid such as an acetate or a lactate or any other anion that is compatible with the ammonium bearing an ester function. The anion X⁻ is more particularly a chloride, a methyl sulfate or an ethyl sulfate.

Use is more particularly made, in the composition according to the invention, of the ammonium salts of formula (IVa) in which:

R₂₂ denotes a methyl or ethyl group,

x and y are equal to 1,

z is equal to 0 or 1,

r, s and t are equal to 2,

R₂₃ is chosen from the group R₂₆—C(═O)—; methyl, ethyl or C₁₄-C₂₂ hydrocarbon-based groups, and a hydrogen atom,

R₂₅ is chosen from the group R₂₈—C(═O)—; and a hydrogen atom,

R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (IVa), mention may be made of the salts, notably the chloride or methyl sulfate of diacyloxyethyldimethylammonium, diacyloxyethylhydroxyethylmethyl ammonium, monoacyloxyethyldihydroxyethylmethylammonium, triacyloxyethylmethylammonium or monoacyloxyethylhydroxyethyldimethylammonium, and mixtures thereof. The acyl groups preferably contain from 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures notably of plant or animal origin, or by transesterification of the methyl esters thereof. This esterification may be followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin. Such compounds are sold, for example, under the names Dehyquart® by the company Henkel, Stepanquat® by the company Stepan, Noxamium® by the company CECA or Rewoquat® WE 18 by the company Evonik.

The composition according to the invention may contain, for example, a mixture of quaternary ammonium monoester, diester and triester salts with a weight majority of diester salts. Use may also be made of the ammonium salts containing at least one ester function that are described in U.S. Pat. Nos. 4,874,554 and 4,137,180. Use may also be made of the behenoylhydroxypropyltrimethylammonium chloride sold, for example, by the company Kao under the name Quartamin BTC 131.

Preferably, the ammonium salts containing at least one ester function contain two ester functions.

According to one preferred embodiment of the invention, the composition (B) comprises one or more cationic surfactants; more preferentially, the cationic surfactant(s) are chosen from cetyltrimethylammonium salts, behenyltrimethylammonium salts and dipalmitoylethylhydroxyethylmethylammonium salts and mixtures thereof; more preferentially from behenyltrimethylammonium chloride or methosulfate, cetyltrimethylammonium chloride or methosulfate, dipalmitoylethylhydroxyethylmethylammonium chloride or methosulfate, and mixtures thereof.

Advantageously, when they are present in the composition (B) according to the invention, the cationic surfactant(s) are present in a total content ranging from 0.5% to 15% by weight, relative to the total weight of the composition (B), and preferably from 1% to 10%.

### Fatty Alcohols

The composition (B) used in step ii) of the process for the treatment of keratin fibres according to the present invention also comprises one or more conditioning agents that can be chosen from fatty alcohols.

The term “fatty alcohol” means a long-chain aliphatic alcohol comprising from 6 to 40 carbon atoms, preferably from 8 to 40 carbon atoms, and comprising at least one hydroxyl group OH.

The fatty alcohols according to the invention are preferably non-oxyalkylenated and non-glycerolated.

The fatty alcohols according to the invention may be saturated or unsaturated, and linear or branched, and preferably include from 8 to 40 carbon atoms.

More preferentially, the fatty alcohols according to the invention are chosen from compounds having the structure R—OH with R denoting a linear or branched, saturated or unsaturated alkyl group optionally substituted with one or more hydroxyl groups, comprising from 8 to 40, better still from 10 to 30, or even from 12 to 24 and even better still from 14 to 22 carbon atoms.

The fatty alcohols may be chosen from solid fatty alcohols and liquid fatty alcohols, and mixtures thereof.

For the purposes of the present invention, the term “solid fatty alcohol” means a fatty alcohol with a melting point of greater than 25° C., preferably greater than or equal to 28° C., more preferentially greater than or equal to 30° C. at atmospheric pressure (1.013×10⁵ Pa).

The solid fatty alcohols may be chosen from saturated or unsaturated, linear or branched solid fatty alcohols, including from 8 to 40 carbon atoms.

The solid fatty alcohols that may be used according to the invention are preferably chosen from compounds having the structure R—OH with R denoting a saturated linear alkyl group optionally substituted with one or more hydroxyl groups, comprising from 8 to 40, better still from 10 to 30, or even from 12 to 24 and even better still from 14 to 22 carbon atoms.

The solid fatty alcohols that may be used may be chosen, alone or as a mixture, from:

lauryl alcohol (or 1-dodecanol);

myristyl alcohol (or 1-tetradecanol);

cetyl alcohol (or 1-hexadecanol);

stearyl alcohol (or 1-octadecanol);

arachidyl alcohol (or 1-eicosanol);

behenyl alcohol (or 1-docosanol);

lignoceryl alcohol (or 1-tetracosanol);

ceryl alcohol (or 1-hexacosanol);

montanyl alcohol (or 1-octacosanol);

myricyl alcohol (or 1-triacontanol).

Preferentially, the fatty alcohol(s) are chosen from cetyl alcohol, stearyl alcohol, behenyl alcohol, myristyl alcohol, and mixtures thereof, such as cetylstearyl alcohol or cetearyl alcohol.

For the purposes of the present invention, the term “liquid fatty alcohol” means a fatty alcohol with a melting point of less than 25° C., preferably less than or equal to 20° C. at atmospheric pressure (1.013×10⁵ Pa).

The liquid fatty alcohols that may be used according to the invention are preferably chosen from compounds having the structure R—OH with R denoting a saturated or unsaturated, linear or branched, preferably unsaturated and/or branched, alkyl group optionally substituted with one or more hydroxyl groups, comprising from 8 to 40, better still from 10 to 30, or even from 12 to 24 and even better still from 14 to 22 carbon atoms.

The liquid fatty alcohols that may be used may be chosen from oleyl alcohol, linoleyl alcohol, linolenyl alcohol, isocetyl alcohol, isostearyl alcohol, 2-octyl-1-dodecanol, 2-butyloctanol, 2-hexyl-1-decanol, 2-decyl-1-tetradecanol and 2-tetradecyl-1-cetanol, and mixtures thereof.

The fatty alcohol(s) according to the invention are advantageously chosen from solid fatty alcohols, and mixtures thereof, preferably from saturated linear solid fatty alcohols including from 8 to 40 carbon atoms, and mixtures thereof.

Preferably, the fatty alcohol(s) are chosen from cetyl alcohol, stearyl alcohol, behenyl alcohol and myristyl alcohol, and mixtures thereof.

According to one preferred embodiment of the invention, the composition (B) comprises one or more fatty alcohols; more preferentially, the fatty alcohol(s) are chosen from cetyl alcohol, stearyl alcohol, behenyl alcohol and myristyl alcohol, and mixtures thereof.

Advantageously, when they are present in the composition (B) according to the invention, the content of the fatty alcohol(s) ranges from 0.5% to 12% by weight, relative to the total weight of the composition (B).

Advantageously, the total content of the conditioning agent(s) chosen from cationic surfactants and fatty alcohols, and mixtures thereof, ranges from 1.5% to 30% by weight, relative to the total weight of the composition (B).

### The Thickeners

The composition (B) used in step ii) of the process for the treatment of keratin fibres according to the present invention can optionally also comprise one or more thickeners.

The thickener(s) may be mineral or organic.

The mineral thickeners may notably be chosen from organophilic clays and fumed silicas, or mixtures thereof.

The organophilic clay may be chosen from montmorillonite, bentonite, hectorite, attapulgite and sepiolite, and mixtures thereof. The clay is preferably a bentonite or a hectorite.

These clays may be modified with a chemical compound chosen from quaternary amines, tertiary amines, amine acetates, imidazolines, amine soaps, fatty sulfates, alkylarylsulfonates and amine oxides, and mixtures thereof.

Organophilic clays that may be mentioned include quaternium-18 bentonites such as those sold under the names Bentone 3, Bentone 38 and Bentone 38V by the company Rheox, Tixogel VP by the company United Catalyst, Claytone 34, Claytone 40 and Claytone XL by the company Southern Clay; stearalkonium bentonites such as those sold under the names Bentone 27 by the company Rheox, Tixogel LG by the company United Catalyst and Claytone AF and Claytone APA by the company Southern Clay; and quaternium-18/benzalkonium bentonites such as those sold under the names Claytone HT and Claytone PS by the company Southern Clay.

The fumed silicas may be obtained by high-temperature hydrolysis of a volatile silicon compound in an oxyhydrogen flame, producing a finely divided silica. This process makes it possible notably to obtain hydrophilic silicas bearing a large number of silanol groups at their surface. Such hydrophilic silicas are sold, for example, under the names Aerosil 130®, Aerosil 200®, Aerosil 255®, Aerosil 300® and Aerosil 380® by the company Degussa, and Cab-O-Sil HS-5®, Cab-O-Sil EH-5®, Cab-O-Sil LM-130®, Cab-O-Sil MS-55® and Cab-O-Sil M-5® by the company Cabot.

It is possible to chemically modify the surface of the silica via chemical reaction in order to reduce the number of silanol groups. It is notably possible to substitute silanol groups with hydrophobic groups: a hydrophobic silica is then obtained.

The hydrophobic groups may be:

trimethylsiloxyl groups, which are notably obtained by treating fumed silica in the presence of hexamethyldisilazane. Silicas thus treated are known as Silica silylate according to the CTFA (6th Edition, 1995). They are sold, for example, under the references Aerosil R812® by the company Degussa, and Cab-O-Sil TS-530® by the company Cabot;

dimethylsilyloxyl or polydimethylsiloxane groups, which are notably obtained by treating fumed silica in the presence of polydimethylsiloxane or dimethyldichlorosilane. Silicas thus treated are known as Silica dimethyl silylate according to the CTFA (6th Edition, 1995). They are sold, for example, under the references Aerosil R972® and Aerosil R974® by the company Degussa, and Cab-O-Sil TS-610® and Cab-O-Sil TS-720® by the company Cabot.

The fumed silica preferably has a particle size that may be nanometric to micrometric, for example ranging from about 5 to 200 nm.

The organic thickeners may notably be chosen from fatty acid amides (coconut monoethanolamide or diethanolamide, oxyethylenated carboxylic acid alkyl ether monoethanolamide), polymeric thickeners such as cellulose-based thickeners (hydroxyethylcellulose, hydroxypropylcellulose or carboxymethylcellulose), guar gum and derivatives thereof (hydroxypropyl guar), gums of microbial origin (xanthan gum, scleroglucan gum), crosslinked acrylic acid or acrylamidopropanesulfonic acid homopolymers and associative polymers (polymers comprising hydrophilic regions and fatty-chain hydrophobic regions (alkyl or alkenyl containing at least 10 carbon atoms) that are capable, in an aqueous medium, of reversibly combining with one another or with other molecules).

Advantageously, when they are present in the composition (B) according to the invention, the content of the thickener(s) ranges from 0.1% to 5% by weight, relative to the total weight of the composition (B).

The composition (B) is preferably aqueous or aqueous-alcoholic.

When the composition (B) is aqueous, it preferably comprises water in a content of greater than or equal to 50% by weight, more preferentially greater than or equal to 70% by weight, even more preferentially still greater than or equal to 90% by weight, relative to the total weight of the composition (B).

When the composition (B) is aqueous-alcoholic, it's can notably comprise one or more organic solvents, preferably in a content ranging from 0.05% to 95% by weight, and more preferentially from 1% to 70% by weight, relative to the total weight of the composition (B).

This organic solvent may be a C₂ to C₄ lower alcohol, in particular ethanol and isopropanol, polyols and polyol ethers such as propylene glycol, polyethylene glycol or glycerol. The organic solvent is preferably ethanol.

### Additives

The compositions (A) and (B) according to the present invention may also optionally comprise one or more additives, other than the compounds of use in the present invention and among which mention may be made of cationic, anionic, non-ionic or amphoteric polymers, or mixtures thereof, antidandruff agents, anti-seborrhoea agents, vitamins and provitamins including panthenol, sunscreens, sequestrants, plasticizers, solubilizers, acidifying agents, alkaline agents, antioxidants, hydroxy acids, fragrances and preserving agents.

Needless to say, those skilled in the art will take care to select this or these optional additional compounds such that the advantageous properties intrinsically associated with the composition according to the invention are not, or are not substantially, adversely affected by the envisaged addition(s).

The above additives may generally be present in an amount comprised, for each of them, of between 0 and 20% by weight relative to the total weight of the detergent composition (A) and/or of the composition (B).

### IMPLEMENTATION OF THE PROCESS

The process for the cosmetic treatment of keratin fibres, in particular human keratin fibres such as the hair, according to the present invention, comprises:

i) a step of applying to said keratin fibres a composition (A) comprising one or more surfactants chosen from anionic surfactants and amphoteric or zwitterionic surfactants, and mixtures thereof.

ii) a step of applying to said keratin fibres a composition (B) comprising one or more compounds of a metal belonging to the group of the rare earth metals, and one or more conditioning agents, preferably chosen from cationic surfactants and fatty alcohols, and mixtures thereof, then

iii) a rinsing step.

According to one preferred embodiment, the composition (B) is applied directly after applying the composition (A), without an intermediate step of rinsing said keratin fibres.

According to the cosmetic treatment process of the invention, the composition (A) is applied before the composition (B).

According to the invention, step ii) is carried out following step i), i.e. step ii) is carried out within a period of time preferably less than or equal to 1 hour after step i), preferably less than or equal to 30 minutes, more preferentially less than or equal to 10 minutes.

According to the invention, step iii) is carried out following step ii), i.e. step iii) is carried out within a period of time preferably less than or equal to 1 hour after step ii), preferably less than or equal to 30 minutes, more preferentially less than or equal to 15 minutes.

According to one particular embodiment, the composition (B) is applied simultaneously with the composition (A), i.e. steps i) and ii) are carried out simultaneously.

The composition (A) according to the invention may be applied to dry or wet keratin fibres, preferably wet keratin fibres.

Preferably, the composition (A) is applied with a leave-on time that may range from 1 to 15 minutes, preferably from 2 to 10 minutes. The composition (A) may be massaged before application of the composition (B).

The composition (B) is then applied to the keratin fibres directly after the application of the detergent composition (A), that is to say without intermediate rinsing of said keratin fibres.

Preferably, the composition (B) is applied with a leave-on time that may range from 1 to 15 minutes, preferably from 2 to 10 minutes.

The composition (A) and the composition (B) present on the keratin fibres may be massaged. The keratin fibres are then rinsed, preferably with water.

The keratin fibres are then optionally dried with a hair-dryer or left to dry naturally.

The present invention also relates to a multi-compartment kit or device comprising a first compartment containing a composition (A) as defined above, and a second compartment containing a composition (B) as defined above.

The examples that follow serve to illustrate the invention without, however, being limiting in nature.

### EXAMPLES

In the examples that follow, all the amounts are given, unless otherwise indicated, as mass percentages of active material relative to the total weight of the composition.

### Example 1

### a) Compositions

The composition (A) was prepared from the ingredients of which the contents are indicated in the table below.

TABLE 1

| | Composition A |
|---|---|
| Sodium laureth sulfate at | 20 |
| 70% in water⁽¹⁾ | |
| Cocoamidomethylbetaine at | 12 |
| 30% in water⁽²⁾ | |
| pH Agent | qs pH 5 |
| Water | qs 100 |

⁽¹⁾sold under the name Texapon N702 by BASF
⁽²⁾sold under the name Dehyton AB 30 by BASF

The composition BO (comparative) and the compositions B1, B2, B3 and B4 (according to the invention) were prepared from the ingredients of which the contents are indicated in the table below:

TABLE 2

| | Compositions B | | | | |
|---|---|---|---|---|---|
| | B0 | B1 | B2 | B3 | B4 |
| Glyceryl stearate⁽³⁾ | 1 | 1 | 1 | 1 | 1 |
| Dipalmitoylethyl- | 5 | 5 | 5 | 5 | 5 |
| hydroxyethyl | | | | | |
| methylammonium | | | | | |
| methosulfate/cetearyl | | | | | |
| alcohol (30/70)⁽⁴⁾ | | | | | |
| Glycerol | 0.8 | 0.8 | 0.8 | 0.8 | 0.8 |
| Cetyltrimethylammonium | 3.5 | 3.5 | 3.5 | 3.5 | 3.5 |
| chloride as an aqueous⁽⁵⁾ | | | | | |
| Yttrium hydroxyacetate | — | 20 | — | — | — |
| Hydrated cerium chloride⁽⁶⁾ | — | — | 20 | — | — |
| Hydrated yttrium chloride⁽⁷⁾ | — | — | — | 20 | — |
| Hydrated lanthanum | — | — | — | — | 20 |
| chloride⁽⁸⁾ | | | | | |
| pH Agent | qs | qs | qs | qs | qs |
| | pH 4 | pH 4 | pH 4 | pH 4 | pH 4 |
| Water | qs 100 | qs 100 | qs 100 | qs 100 | qs 100 |

⁽³⁾sold under the name Tegomul 6070 by Evonik Goldschmidt
⁽⁴⁾sold under the name Dehyquart F30 by BASF
⁽⁵⁾sold under the name Dehyton A OR by BASF
⁽⁶⁾sold under the name CeCl₃ · 7H₂O by Sigma-Aldrich
⁽⁷⁾sold under the name YCl₃ · 6H₂O by Sigma-Aldrich
⁽⁸⁾sold under the name LaCl₃ · 7H₂O by Sigma-Aldrich

### b) Procedure

20 g of locks of bleached hair (Platifiz) (of 2.7 g) are shampooed by applying to wet hair 10 g of composition A with a leave-on time of 5 minutes.

Then, with or without intermediate rinsing, 10 g of composition B0, B1, B2, B3 or B4 are applied with a leave-on time of 10 minutes.

According to the process of the invention, after application of the composition B (B0, B1, B2, B3 or B4), the locks are rinsed under a stream of water for 6 seconds at 10 l/min, that is to say a volume of water of 1 l.

The cosmetic properties of the hair are then evaluated by evaluating the glidant feel of the locks, which is representative of the presence or absence of the compositions A and/or B, on a scale of 0 to 5, with:

0: no glidant effect, the hair is perfectly free of the compositions A and/or B,

5: strong presence of residues on the hair, requiring a need to continue rinsing.

The rinsing is then continued. The moment when the hair is sufficiently free of residues to be able to stop the stream of water is thus evaluated by passing the hair under the fingers. The total amount of water used is then noted.

The hair is then disentangled, while noting the ease with which a comb passes through it, on a scale of 0 to 10, with: 0=impossible to disentangle, 10: very easy to disentangle.

Then, once the hair is disentangled, it is dried with a hair-dryer using the hot air flow. The lightness of the hair is then noted by evaluating by feel whether the hair forms a compact whole (grade=0) or, on the contrary, an assembly of total individualized strands of hair (grade=5).

### c) Results

The results of the various evaluations are presented in the table below:

TABLE 3

| Protocol | | | | Evaluation | | | | |
|---|---|---|---|---|---|---|---|---|
| | | | | Cosmetic | | | Disen- | |
| | | | | properties after | | Total | tangling of | Lightness |
| | | Intermediate | Com- | passing under | Complete | amount | the wet hair | of the |
| | | rinsing | position | a 1 l stream | rinsing | of water | after rinsing | dry hair |
| | | (5 min after | B | of water | (in litres | used* (in | (grade 0 | (grade 0 |
| Test | | shampooing) | (10 min) | (grade 0 to 5) | of water) | litres) | to 10) | to 5) |
| Test | Composition | No | B1 | 2 | 5 | 5 | 9 | 4 |
| No. 1 | A | | | | | | | |
| Test | Composition | No | B2 | 2 | 6.5 | 6.5 | 9 | 4 |
| No. 2 | A | | | | | | | |
| Test | Composition | No | B3 | 1 | 5.1 | 5.1 | 9 | 4.5 |
| No. 3 | A | | | | | | | |
| Test | Composition | No | B4 | 1 | 5.8 | 5.8 | 7 | 4 |
| No. 4 | A | | | | | | | |
| Test | Composition | No | B0 | 5 | 8.9 | 8.9 | 3 | 3.7 |
| No. 5 | A | | | | | | | |
| Test | Composition | Yes | — | — | — | 4.5 | 2.1 | 4.25 |
| No. 6 | A | | | | | | | |
| Test | Composition | Yes | B0 | 2.5 | 5 | 9.5 | 9 | 2 |
| No. 7 | A | | | | | | | |

*The total amount of water used represents the amount of water used during the optional intermediate rinsing, during the passing under a 1 l stream of water and during the complete rinsing.

In test No. 7, in which a shampooing operation (composition A), a rinsing operation, a care treatment without rare earth metal (composition BO) and then a rinsing operation are carried out, the total amount of water required to obtain complete rinsing is 9.5 l. The hair is easy to disentangle, but is not very well separated into individual strands and does not therefore have a light appearance.

In test No.5, in which a shampooing operation (composition A), a care treatment without rare earth metal (composition BO) without intermediate rinsing and then a final rinsing operation are carried out, the hair has a considerable glidant feel (grade of 5) and it is necessary to rinse the hair abundantly in order to completely rid it of the residues of the compositions A and BO. Despite the absence of intermediate rinsing, the total amount of water required in this test in order to obtain complete rinsing is large (8.9 l). Furthermore, disentangling of the hair is difficult; it is comparable to that observed in test No. 6 in which the hair is subjected to a shampooing operation (composition A), but not a care treatment with a composition B.

In tests Nos. 1 to 4, in which a shampooing operation (composition A), a care treatment comprising a rare earth metal (composition B1, B2, B3 or B4) without intermediate rinsing and then a final rinsing operation are carried out, the hair has a feel that is not very glidant after passing under a 1 l stream of water. The amount of water required for complete rinsing, and therefore the total amount of water used, is therefore reduced (between 5 l and 6.5 l). Furthermore, the hair can be very easily disentangled after rinsing and the strands of hair are well separated after drying, giving the head of hair a light appearance.

Thus, the process according to the invention, using a shampoo comprising at least one surfactant chosen from anionic and/or amphoteric and zwitterionic surfactants, and a composition comprising a rare earth metal in the +III oxidation state, makes it possible to obtain the best results in terms of water saving and a significant care effect, in particular in terms of disentangling and lightness of the hair.

### Example 2

The same test as in Example 1 is carried out by repeating the protocol 3 times.

The results of the various evaluations are presented in the table below:

TABLE 4

| Protocol carried out 3 times | | | | Evaluation carried out at the time of the 3rd protocol | | | | |
|---|---|---|---|---|---|---|---|---|
| | | | | Cosmetic | | | Disen- | |
| | | | | properties after | | | tangling of | Lightness |
| | | Intermediate | | passing under | Complete | Total | the wet hair | of the |
| | | rinsing | | a 1 l stream | rinsing | amount of | after rinsing | dry hair |
| | | (5 min after | Comp B | of water | (in litres | water used* | (grade 0 | (grade 0 |
| Test | | shampooing) | (10 min) | (grade 0 to 5) | of water) | (in litres) | to 10) | to 5) |
| Test | Comp A | No | B1 | 1.5 | 4.5 | 4.5 | 9 | 4.25 |
| No. 8 | | | | | | | | |
| Test | Comp A | No | B2 | 1.5 | 5.5 | 5.5 | 9 | 4 |
| No. 9 | | | | | | | | |
| Test | Comp A | No | B3 | 0.75 | 4 | 4 | 9 | 4.75 |
| No. 10 | | | | | | | | |
| Test | Comp A | No | B4 | 1 | 5 | 5 | 8 | 4.25 |
| No. 11 | | | | | | | | |
| Test | Comp A | No | B0 | 5 | 9.5 | 9.5 | 3 | 3.25 |
| No. 12 | | | | | | | | |
| Test | Comp A | Yes | — | — | — | 5 | 1.75 | 4.25 |
| No. 13 | | | | | | | | |
| Test | Comp A | Yes | B0 | 2.75 | 5.5 | 10 | 9 | 0.75 |
| No. 14 | | | | | | | | |

*The total amount of water used represents the amount of water used during the optional intermediate rinsing, during the passing under a 1 l stream of water and during the complete rinsing.

In test No. 4, in which a shampooing operation (composition A), a rinsing operation, and a care treatment without rare earth metal (composition BO), this protocol being repeated three times, and then a rinsing operation are carried out, the total amount of water required to obtain complete rinsing is 10 l. The hair is easy to disentangle, but is not very well separated into individual strands and does not therefore have a light appearance.

In tests Nos. 8 to 10, in which a shampooing operation (composition A), and a care treatment comprising a rare earth metal (composition B1, B2, B3 or B4) without intermediate rinsing, this protocol being repeated three times, and then a final rinsing operation are carried out, the hair has a feel that is not very glidant after passing under a 1 l stream of water. The amount of water required for complete rinsing, and therefore the total amount of water used, is therefore reduced (between 4 l and 5.5 l). Furthermore, the hair can be very easily disentangled after rinsing and the strands of hair are well separated after drying, giving the head of hair a light appearance.

It is also noted that the repeating of the protocol in tests Nos. 8 to 10 makes it possible to increase the satisfaction, notably in terms of lightness of the hair, compared to tests Nos. 1 to 4. The repeating of the protocol also makes it possible to obtain a greater water saving than that observed in tests Nos. 1 to 4 above.

Repeated applications therefore make it possible to increase the advantages.

### Example 3

### a) Compositions

The compositions G1 and G2 were prepared from the ingredients of which the contents are indicated in the table below:

TABLE 5

| | Compositions G | |
|---|---|---|
| | G1 | G2 |
| Glyceryl stearate⁽³⁾ | 1 | 1 |
| Dipalmitoylethylhydroxyethyl methylammonium | 5 | 5 |
| methosulfate/cetearyl alcohol (30/70)⁽⁴⁾ | | |
| Glycerol | 0.8 | 0.8 |
| Cetyltrimethylammonium chloride as an aqueous | 3.5 | 3.5 |
| solution⁽⁵⁾ | | |
| Yttrium hydroxyacetate | 6.7 | |
| Hydrated cerium chloride⁽⁶⁾ | — | 6.7 |
| Hydrated yttrium chloride⁽⁷⁾ | — | — |
| Hydrated lanthanum chloride⁽⁸⁾ | — | — |
| pH Agent | qs pH 4 | qs pH 4 |
| Water | qs 100 | qs 100 |

⁽³⁾sold under the name Tegomul 6070 by Evonik Goldschmidt
⁽⁴⁾sold under the name Dehyquart F30 by BASF
⁽⁵⁾sold under the name Dehyton A OR by BASF
⁽⁶⁾sold under the name CeCl₃ · 7H₂O by Sigma-Aldrich
⁽⁷⁾sold under the name YCl₃ · 6H₂O by Sigma-Aldrich
⁽⁸⁾sold under the name LaCl₃ · 7H₂O by Sigma-Aldrich

### b) Results

Locks are treated according to the protocol of Example 2, then treated with the protocol detailed in Table 6, that is to say by again repeating the protocol 3 times.

The results of the various evaluations are presented in the table below:

TABLE 6

| Protocol carried out 3 times | | | | Evaluation carried out at the time of the 3rd protocol | | | | |
|---|---|---|---|---|---|---|---|---|
| | | | | Cosmetic | | | Disen- | |
| | | | | properties after | | Total | tangling of | Lightness |
| | | Intermediate | Com- | passing under | Complete | amount | the wet hair | of the |
| | | rinsing | position | a 1 l stream | rinsing | of water | after rinsing | dry hair |
| | | (5 min after | B | of water | (in litres | used* (in | (grade 0 | (grade 0 |
| Test | | shampooing) | (10 min) | (grade 0 to 5) | of water) | litres) | to 10) | to 5) |
| Test | Com- | No | G1 | 2 | 5.5 | 5.5 | 8.5 | 4 |
| No. 15 | position A | | | | | | | |
| Test | Com- | No | G2 | 2.75 | 6.5 | 6.5 | 8 | 3 |
| No. 16 | position A | | | | | | | |
| Test | Com- | No | B3 | 1.5 | 5 | 5 | 9 | 4.25 |
| No. 17 | position A | | | | | | | |
| Test | Com- | No | B4 | 2 | 5.5 | 5.5 | 8 | 4 |
| No. 18 | position A | | | | | | | |
| Test | Com- | No | B0 | 5 | 9.5 | 9.5 | 3 | 3.25 |
| No. 19 | position A | | | | | | | |
| Test | Com- | Yes | — | — | — | 5 | 1.75 | 4.25 |
| No. 20 | position A | | | | | | | |
| Test | Com- | Yes | B0 | 2.75 | 5.5 | 10 | 9 | 0.75 |
| No. 21 | position A | | | | | | | |

*The total amount of water used represents the amount of water used during the optional intermediate rinsing, during the passing under a 1 l stream of water and during the complete rinsing.

The disentangling and the rinsing waters are noted after the 3 applications of the protocol. It is observed that, despite a reduced amount of rare earth metal, the advantages detected above are kept.

### Example 4

### a) Composition

The composition E was prepared from the ingredients of which the contents are indicated in the table below:

TABLE 7

| | Composition E |
|---|---|
| Cocoglucoside at 50% in water⁽⁹⁾ | 20 |
| Water | qs 100 |

⁽⁹⁾sold under the name Plantacare 818 UP by BASF

### b) Procedure

20 g of locks of bleached hair (Platifiz) (of 2.7 g) are shampooed by applying to wet hair 10 g of composition E with a leave-on time of 5 minutes.

Then, with or without intermediate rinsing, 10 g of composition B3 as described above are applied with a leave-on time of 10 minutes.

According to the process of the invention, after application of the composition E, the locks are rinsed with a volume of water of 5 l.

### c) Results

Hair that is easy to disentangle, and has a clean appearance once dry, with a pronounced care effect and a texture effect is obtained.

1. Process for the cosmetic treatment of keratin fibres, comprising:
i) a step of applying to said keratin fibres a composition (A) comprising one or more surfactants chosen from anionic surfactants and amphoteric or zwitterionic surfactants, and mixtures thereof.
ii) a step of applying to said keratin fibres a composition (B) comprising one or more compounds of a metal belonging to the group of the rare earth metals, and one or more conditioning agents chosen from cationic surfactants and fatty alcohols, and mixtures thereof, then
iii) a step of rinsing said keratin fibres.

2. Process according to claim 1, characterized in that said anionic surfactant(s) are chosen from sulfates, sulfonates and carboxylic surfactants, and mixtures thereof preferably from C₆-C₂₄ alkyl sulfates, C₆-C₂₄ alkyl ether sulfates, C₆-C₂₄ alkyl sulfosuccinates, C₆-C₂₄ alkyl ether sulfosuccinates, (C₆-C₂₄)acylisethionates, C₆-C₂₄ acylsarcosinates, (C₆-C₂₄)alkyl ether carboxylates, polyoxyalkylenated (C₆-C₂₄)alkyl (amido) ether carboxylic acids and salts thereof, C₆-C₂₄ acylglutamates, C₆-C₂₄ acylglycinates, and mixtures thereof.

3. Process according to claim 1, characterized in that said amphoteric or zwitterionic surfactant(s) are chosen from (C₈-C₂₀)alkylbetaines, (C₈-C₂₀)alkylsulfobetaines, (C₈-C₂₀)alkylamido(C₃-C₈)alkylbetaines, (C₈-C₂₀)alkylamido(C₆-C₈)alkylsulfobetaines, and the compounds of formula (3):
Rₐ—CONHCH₂CH₂—N⁺(R_{b})(R_{c})—CH₂COO⁻,M⁺,X⁻
in which:
Rₐ represents a C₁₀ to C₃₀ alkyl or alkenyl group derived from an acid RaCOOH;
R_{b} represents a β-hydroxyethyl group; and
R_{c} represents a carboxymethyl group;
M⁺ represents a cationic counterion derived from an alkali metal or alkaline-earth metal, an ammonium ion or an ion derived from an organic amine; and
X⁻ represents an organic or mineral anionic counterion, preferably chosen from halides, acetates, phosphates, nitrates, (C₁-C₄)alkyl sulfates, (C₁-C₄)alkyl- or (C₁-C₄)alkylarylsulfonates; or alternatively M⁺ and X⁻ are absent;
and mixtures thereof.

4. Process according to claim 1, characterized in that the content of said surfactant(s) chosen from anionic surfactants and amphoteric or zwitterionic surfactants, and mixtures thereof, is greater than or equal to 4% by weight, preferably ranges from 4% to 35% by weight, relative to the total weight of the detergent composition (A).

5. Process according to claim 1, characterized in that the metal of the group of the rare earth metals is chosen from cerium, yttrium, ytterbium, lanthanum and europium.

6. Process according to claim 1, characterized in that the metal belonging to the group of the rare earth metals is in the +III oxidation state.

7. Process according to claim 1, characterized in that the metal compound(s) are chosen from rare earth metal salts, rare earth metal complexes or rare earth metal oxides.

8. Process according to claim 1, characterized in that the metal compound(s) are chosen from compounds combined with a monoatomic or non-monoatomic monoanionic ion, compounds combined with a di- or trianionic ion, compounds combined with a ligand comprising one or more groups forming a coordination bond and a function forming an ionic bond, rare earth metal oxides, and mixtures thereof preferably from yttrium dihydroxyacetate, cerium chloride, yttrium chloride, lanthanum chloride, Ce(acetonate)3, cerium oxide, lanthanum oxide, yttrium oxide, erbium oxide, scandium oxide, ytterbium oxide and europium oxide, and mixtures thereof.

9. Process according to claim 1, characterized in that the content of said metal compound(s) is greater than or equal to 0.1% by weight, preferably greater than or equal to 0.25% by weight, more preferentially ranges from 0.5% to 20% by weight, relative to the total weight of the composition (B).

10. Process according to claim 1, characterized in that said cationic surfactant(s) of the composition (B) are chosen from cetyltrimethylammonium salts, behenyltrimethylammonium salts and dipalmitoylethylhydroxyethylmethylammonium salts and mixtures thereof; more preferentially from behenyltrimethylammonium chloride or methosulfate, cetyltrimethylammonium chloride or methosulfate, dipalmitoylethylhydroxyethylmethylammonium chloride or methosulfate, and mixtures thereof.

11. Process according to claim 1, characterized in that said fatty alcohol(s) of the composition (B) are chosen from cetyl alcohol, stearyl alcohol, behenyl alcohol, myristyl alcohol, and mixtures thereof.

12. Process according to claim 1, characterized in that the total content of said conditioning agent(s) chosen from cationic surfactants and fatty alcohols, and mixtures thereof, ranges from 1.5% to 30% by weight, relative to the total weight of the composition (B).

13. Process according to claim 1, characterized in that the composition (B) also comprises one or more thickeners, preferably chosen from thickeners of mineral or organic origin, more preferentially from organophilic clays and fumed silicas, or mixtures thereof.

14. Process according to claim 1, characterized in that the composition (B) is applied directly after applying the composition (A), without an intermediate step of rinsing the keratin fibres.

15. Multi-compartment kit or device comprising a first compartment containing a detergent composition (A) as defined according to claim 1, and a second compartment containing a composition (B).
